(19) United States
(12) Patent Application Publication
(10) Pub. No.: US 2009/0204067 A1
(43) Pub. Date: Aug. 13, 2009

(54) Two-part percutaneous endoscopic intragastric surgery cannula

(76) Inventor: Mohamed Saad Abu-Halawa, Jeddah (SA)

Correspondence Address: Dr. Mohamed Saad;Alzahraa Hospital, Prince Sultan Street, PO BOX 15751, Jeddah, 21454, SA

(21) Appl. No.: 12/012,865

(22) Filed: Feb. 7, 2008

(51) Int. Cl. A61B 17/94; A61B 17/02; A61B 17/34

(52) U.S. Cl. 604/96.01; 604/256

(57) ABSTRACT

An apparatus for accessing the gastric lumen, allowing elaborate intragastric surgeries to be carried on. The apparatus consists of two parts; an external part and a transgastric part. The named external part is a cylinder ending in a larger part of different shapes, the external part will be equipped with a mechanism to allow instruments to get inside the stomach while preventing gas from escaping out. It will also contain a gas delivery port to allow insufflation of the stomach through the cannula. The named transgastric part consists of a cylinder of variable lengths ending in an internal gastric bolster that will be either solid-type or balloon-type. This part is attached to a tapering introduction system ending in a wire loop to be used for introduction of this part. The apparatus also includes an extraction catheter to be used during removal of transgastric part with solid-type gastric bolster.

### TECHNICAL FIELD

The present invention relates to medical devices and procedures, and more particularly to apparatus and methods for performing percutaneous intrasgastric minimally invasive surgical procedures. It may also be of application to those parts of colon that could be accessed through both endoscope and the skin.

### BACKGROUND OF INVENTION AND THE PERVIOUS ART

Minimally invasive surgery has already proved its value in the medical practice. Laparoscopic surgeons are now performing an increasing number of operations that were traditionally done by open surgical methods. This advance entails a huge technical advance in instruments and laparoscopic equipment. However, transfer of these experiences and technologies to intraluminal surgery like intragastric surgery was much slower than hoped.

This lagging of intragastric surgery has many reasons, including the need for special instruments, the difficult orientation, the risk of sepsis, the physiological effect and most importantly dealing with gastric perforation at the end of operation. In 1995 Filipi et used standard PEG (precutaneous endoscopic gastrostomy) tube as gastric access device. At the end of the operation they pulled it out and repaired the gastric defect through minilaparotomy.

In 1997 Stefan B, et al introduced what they called gastrotrocar that consist of a 7 mm polyethylene tube that is passed through the stomach then a valve is screwed onto the tube after it emerges from the abdominal wall. The group used their trocar to treat pancreatic cysts. At the end of their procedure the trocar was kept in place and only removed later after maturation of the tract several days later. Both methods didn't address the problem of removal of the gastric access device as the former authors did laparotomy for this step and the later waited for maturation of tract before pulling the device out.

Although these models could be snared by a endoscopic snare and pulled out together with the scope (as they are too big to be pulled through the biopsy channels). During that time there may be bleeding from gastric perforation, escape of gastric air or gastric fluid to the peritoneal cavity.

In a recent invention (US 2007/0156165 A1), a device for accessing the stomach cavity was designed in such a away to be passed from the mouth through the stomach wall and then through the skin. According to the inventors' description, at the end of the operation, the port is removed by deflating an internal bumper and simply pulling the port through the skin. To achieve the goal of having a port that will pass through the mouth and get out through the skin, the inventor designed a short port (2.5-4 cm) with an inflatable bumper.

The problems with this design are:
1-As the port length is described to range from 2.5-4 cm, that length will be just adequate to extend from the stomach wall to the skin if used in a human, (which is clearly the inventors' intention) which will make it extremely difficult for surgeons to handle the port; as to inflate the internal bumper, attach gas for stomach insufflation or to attach an external bumper. It is easy to imagine how difficult the manipulation of such a short port will be and how easy it could slip in or out.
2-What will stop the trocar from slipping from the stomach is an inflatable bumper that will be inflated after the dilating introducing portion comes out of the skin and removed as it is not possible to inflate the bumper through a removable part. Also considering that port's length rang from 2.5-4 cm. It is highly doubted that such a short cylindrical tube can remain in position for any useful time to allow insufflation of the bumper and what we positively expect is that the stomach will slide back leaving the trocar free in the peritoneal cavity while the hole in the stomach (5-15 mm in the design) will either leak gastric content to the peritoneal cavity or bleed or worse still, both.
3-Also incorporating a bumper inflation system, a gas delivery system and a gas leakage control system in that port will certainly result in increase in the minimal diameter that could be manufactured of this port and will certainly result in increased external diameter of the port and hence the resultant gastric hole needed to pass it In conclusion that port is too short, difficult to handle, complex to manufacture and carries a very high risk of slippage in or out.

### SUMMARY OF THE INVENTION

Broadly our invention provides an apparatus and method that can help surgeons to access the stomach interior in a minimally invasive way, allowing them to do a lot of invasive interventions in a practical, easy and safe way.

A main feature of our cannula is that we divided it into two parts, an external part and a transgastric part. The latter is introduced through the mouth while the external part (which can be as large as needed) is attached to the transgastric part after its proximal end comes through the skin.

In this way we can have a cannula that is exactly like the ordinary laparoscopic trocar which will allow passage of almost all available laparoscopic instruments and those which could be invented specifically for this surgical approach. The external part will be equipped with a gas delivery system and a gas leakage control system, contrary to the pervious invention, this will add nothing to the bulk of the transgastric part of the cannula.

The transgastric part will be secured in the stomach by an internal gastric bolster that will act as a brake to prevent outward movement, no inflation needed and no chance of problematic slippage of the cannula. That bolster can either be solid-type or a balloon-type, the later will not have inflation-deflation mechanism, it will come already inflated and will be ruptured endoscopically at the end of the procedure, thus will not again add any bulk to the cylindrical part of the transgastric part. In addition an external bolster will be fixed around the transgastric part of the cannula that is further fixed to the skin by an adhesive sheet to prevent inward movement of the cannula.

The transgastric part will be longer than the length of previous design to allow comfortable and safe handling of the trocar.

The removal method of the cannula at the end of the procedure will differ depending on the type of internal gastric bolster used. For cannulas with solid-type internal gastric bolster, the cannula will be disassembled and cold snare introduced endoscopically to encircle the transgastric part of the cannula at the junction of the cylindrical part with the internal gastric bolster, then an extraction catheter with an inflatable balloon is passed through the transgastric part that is then removed through the mouth while the balloon of the extraction catheter is inflated and used to maintain gastric wall traction until the endoscope is reintroduced and an endoscopic clip passed to close the resultant gastric defect at the same time the extraction catheter is removed.

The use of this extraction catheter during this time will maintain gastric insufflation and prevent leakage of gastric contents or gastric wall bleeding during the time the endoscope is removed with the transgastric part of the cannula. This will be of greater necessity with the larger diameters' cannulas.

On the other hand cannulas with balloon-type internal gastric bolster will be removed by rupturing the balloon using suitable endoscopic needle and removing the cannula through the skin. So, whether cannula has solid-type or balloon-type gastric bolster, removal of the cannula will be easy and safe. This an important improvement feature in our design

### BRIEF DESCRIPTION OF THE DRAWINGS

FIG. 1 is a horizontal sectional view of the named external part of the PEIGS cannula according to one aspect of the invention;

FIG. 2 is a sagittal sectional view of the named external part of the PEIGS cannula showing the gas inlet according to an embodiment of the invention;

FIG. 3 is the named transgastric part of the PEIGS cannula according to the invention showing the solid-type internal gastric bolster that will prevent slipping of the cannula outside the stomach according;

FIG. 4. is the named transgastric part of the (PEIGS) cannula with the balloon-type internal gastric bolster, this balloon is filled with inert fluid material.

FIG. 5 is the external bolster that will fix the cannula to the skin aided by a adhesive sheet to prevent axial movement of the cannula according to the invention;

FIG. 6 shows the named transgastric part of the PEIGS cannula attached to a long tapering introducing system ending in a wire loop;

FIG. 7 shows the assembled PEIGS cannula in situ according to an embodiment of the invention.

FIG. 8 shows the extraction catheter with its balloon deflated, passing inside the transgastric part of the (PEIGS) cannula after removing the external part while a snare is encircling the junction between the cylindrical part and the internal gastric bolster in preparation for removal of transgastric part through the mouth. This will be needed to remove cannulas with solid-type internal gastric bolster.

FIG. 9 shows the extraction catheter with the balloon inflated keeping the stomach retracted towards the abdominal wall while sealing the gastric defect as the endoscopist is preparing to deploy his endoclips.

FIG. 10 shows the assembled cannula with a balloon-type internal gastric bolster in situ, with the endoscopist ready to rupture the balloon with an endoscopic needle in preparation for its removal through the skin.

### DESCRIPTION OF THE INVENTION

The following detailed description is not to be taken in a limiting sense, it is made merely for the purpose of illustrating the general principles of the invention, while the scope of the invention is best defined by the appended claims.

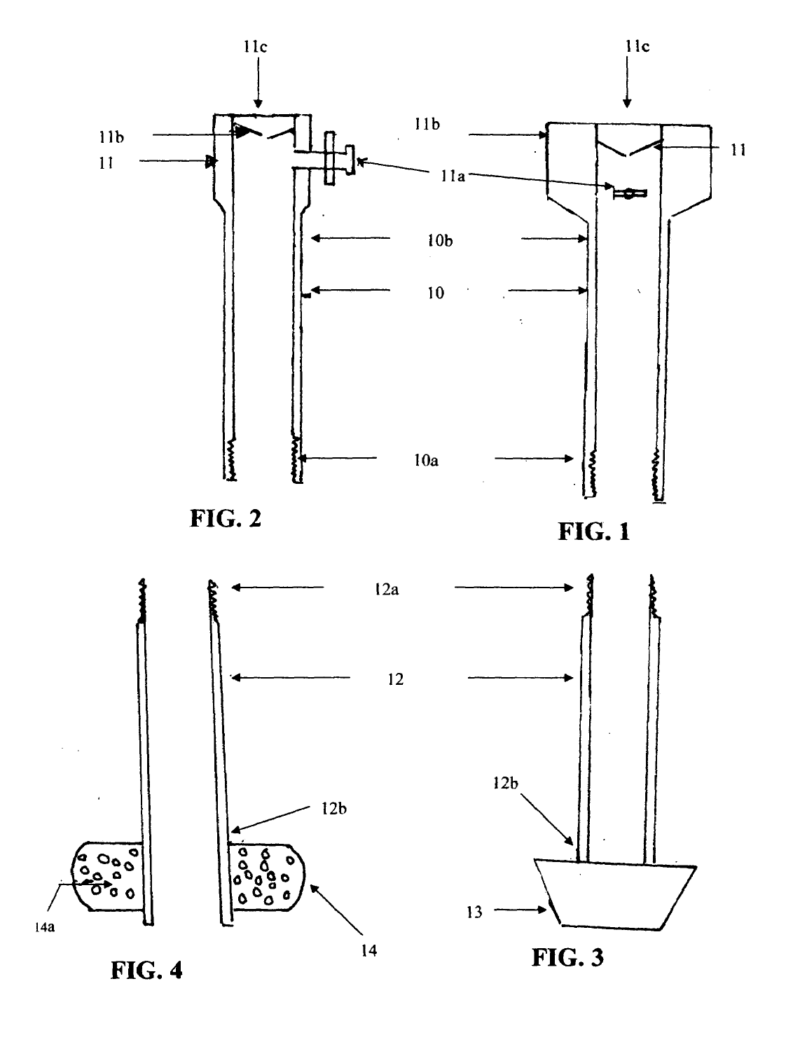

FIGS. 1 & 2 are sectional views of the external part of the percutenous endoscopic intragastric surgery (PEIGS) cannula according to one embodiment of the present invention. The apparatus comprises a cylindrical tube (10) that has a distal end (10a) and a proximal end (10b). The distal end is adapted to attach to the transgastric part of the percutenous endoscopic intragastric surgery (PEIGS) cannula (FIG. 3). The method of attachment and hence the design of that segment could vary, but regardless of the mechanism used it has to ensure a secure airtight attachment and at the same time maintain smooth and regular internal surface. The internal diameter of that tube (10) should match the diameter of the transgastric part of the percutenous endoscopic intragastric surgery (PEIGS) cannula. The length of that cylindrical part will be kept to the minimum needed to ensure comfortable handling of the assembled cannula without being unnecessarily too long.

The proximal end (10b) is attached to a dilated part (11), which can take variable shapes. According to one embodiment of the present invention, that dilatation will facilitate handling of the assembled cannula and will contain a gas inlet (11a) to allow percutaneous insufflation of the stomach and a mechanism to prevent gas escape while allowing surgical instruments of compatible diameter to go in and out through its proximal opening (11c). The external part of the percutenous endoscopic intragastric surgery (PEIGS) cannula can be made from plastic or metal. If it is made of metal then that part of the cannula can be reusable according to another embodiment of the invention.

FIGS. 3 & 4 is a sectional views of the transgastric part of the percutenous endoscopic intragastric surgery (PEIGS) cannula according to one aspect of the present invention. The apparatus comprises a cylindrical tube (12) that has a proximal end (12a) and a distal end (12b). The proximal end (12a) is adapted to attach to the external part of the percutenous endoscopic intragastric surgery (PEIGS) cannula (FIGS. 1 & 2). The method of attachment and hence the design of that segment could vary, but regardless of the mechanism used, it has to ensure a secure airtight attachment and at the same time maintain smooth and regular internal surface. The distal end of this cylinder (12b) is attached to an internal gastric bolster (13 & 14) that will prevent the slippage of the tube from the stomach during insertion of the transgastric part of the (PEIGS) cannula as well as during conduct of intragastric surgery. We developed two designs for this segment In one design this part will be made from solid plastic-like material (13), the consistency of this part will be soft and deformable to make extraction through the mouth easy at the end of the operation. It can be of different shapes, as an example it may take the shape of a tulip end.

In the other design that bolster will be made of a thin walled balloon (14) filled with inert fluid (14a), that balloon will be ruptured at the end of the operation by an endoscopic needle and the cannula will be simply removed through the skin.

The solid-type internal gastric bolster will fix the cannula in a secure way but will need the transgastric part to be removed through the mouth in an extra step. One the other hand the balloon-type bolster will make cannula removal easier but will carry the risk of rupture during insertion and/or the procedure. Detailed animal experiments and human studies will determine the more suitable design.

The cylindrical part (12) can be made of variable substances like plastic, but regardless of the material used, that part should retain enough stiffness to allow smooth passage of instruments in and out and to retain straight alignment of the cannula.

The internal diameter of this part of the cannula (12) can range from 2 mm to 15 mm depending on the intended intervention while the internal gastric bolster (13 & 14) will be of larger diameter (at least twice the diameter of the cylindrical part) to allow secure position of the cannula.

By using (PEIGS) cannulas with an internal diameter of 2-3 mm, needlescopic percutaneous intragastric surgery could be performed according to another embodiment of the present invention.

The length of the cylindrical part according to the invention will range from 5 cm to 8 cm but may be more or less depending on the patient's body built and hence the thickness of his abdominal and gastric wall. The ideal length of that part of the cannula will equal the estimated combined abdominal wall and gastric wall thickness plus 3-4 cm. This 3-4 cm segment will protrude outside the skin after insertion of the transgastric part and will allow comfortable handling of that part of the cannula to allow safe and easy fixation of the external bolster and cannula assembly.

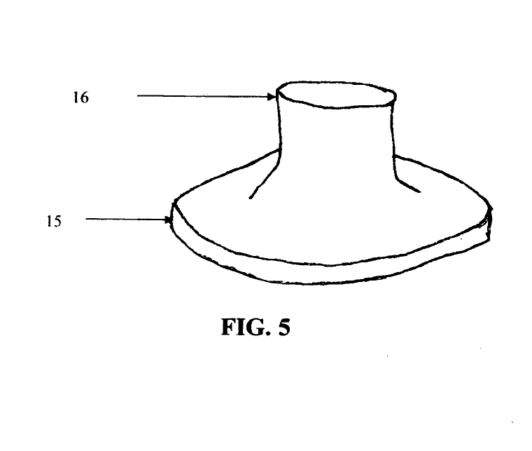

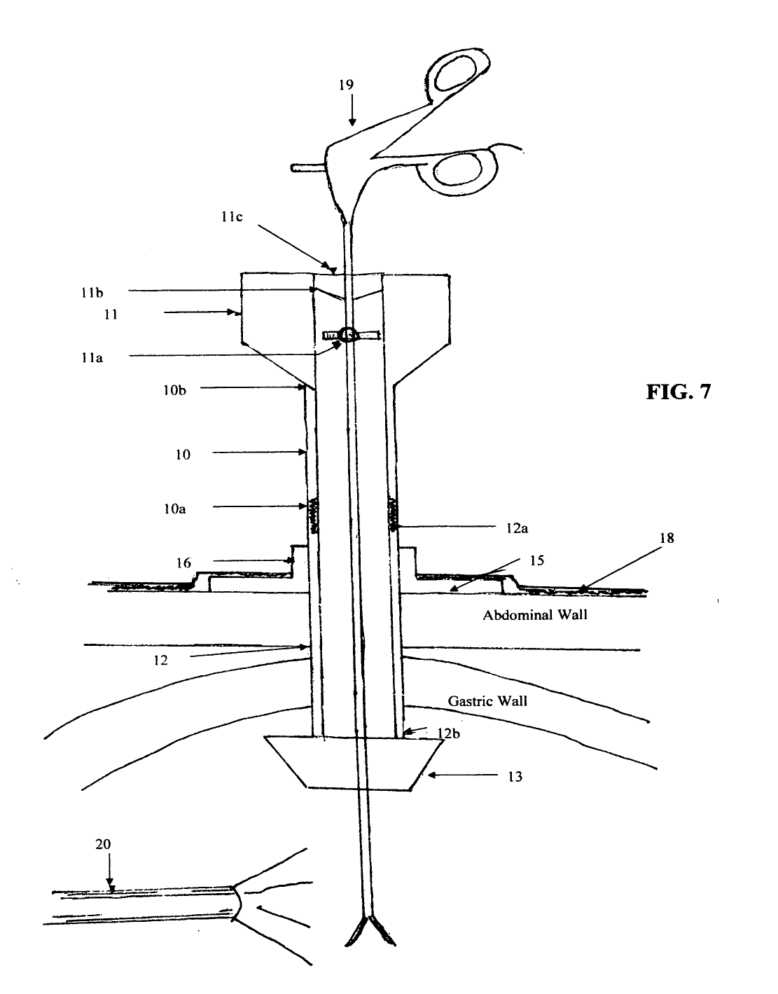

FIG. 5 is a schematic representation of the external bolster that will be fixed around the cylindrical part of the transgastric part of the PEIGS cannula (12) to prevent the axial movement of the cannula. The shown example compromises 2 parts, a short cylindrical part (16) with internal diameter to fit with the transgastric part of the PEIGS. The other part (15) is a wide thin part that will rest on the patient's skin to prevent axial movement of the cannula. This part will be fixed to the skin by either incorporating adhesive material to that part (15) itself or by fixing it by a separate adhesive sheet (18) as shown in FIG. 7. However, the design of this bolster may vary, as for example the two components (15 & 16) can be made separately, or a hinge could be included in the bolster, the aim of these modifications is to allow easier fixation of it around the cannula without struggling, as this may predispose to slipping of the transgastric part of the PEIGS cannula inside the stomach.

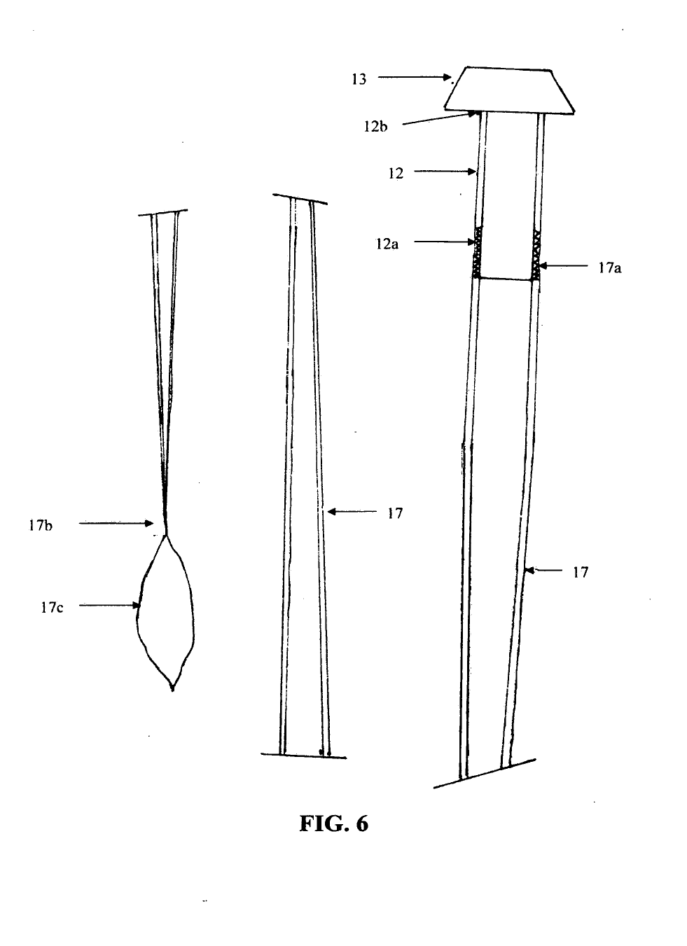

FIG. 6 This is a sectional view of the transgastric part of the PEIGS cannula mounted on the introduction system (17) according to an embodiment of the present invention, this is a tapering system that has a wider distal end (17a) attached to the proximal end of the transgastric part of the PEIGS cannula. This attachment can have different designs but in all cases must be secure enough to withstand traction needed to pass the system through the gastric and abdominal walls. The other end of the introduction system is rather tapered (17b) and is attached to a wire loop (17c).

According to an aspect of the invention the transgastric part of the PEIGS trocar can be introduced by passing a needle through the skin to puncture the endoscopically inflated stomach. A guide wire is then passed through the needle to be grasped by a snare or other suitable endoscopic instrument and extracted through the mouth. This wire will be attached securely to the wire loop of the introduction system (16c ). Then by pulling the guide wire the introduction system will pass through the mouth, to the stomach then through the stomach and abdominal walls to exit through a small skin incision. This will finally allow passage of the transgastric part of the PEIGS cannula.

Once this part of the cannula is outside the skin the introduction system is removed, the transgastric part of the PEIGS cannula is pulled gradually and gently till the stomach wall is in contact with the abdominal walls. During this maneuver the internal gastric bolster will prevent slippage of the cannula unless excessive force is used. The external bolster is then attached around the cannula and further secured by adhesive sheet to the skin, then the external part of the cannula is attached to assemble the complete PEIGS cannula. Other ways of passing transgastric part of the PEIGS cannula could be done and a push method over a similar guide wire could be equally effective according to an embodiment of the invention.

FIG. 7 is a sectional view through the fully assembled and fixed PEIGS cannula in situ traversing the gastric and the abdominal wall with an instrument (19) passing through the cannula to the stomach so that interventions can be done under endoscopic (20) control.

It would be possible to use almost all laparoscopic tools as for example monopolar and bipolar cautery, harmonic scalpel, powerful suction irrigation and staplers in managing stomach lesions. Other instruments in current use for laparoscopic surgery could also be used. It is also expected that specially designed instruments for intragastric surgery adapted for stomach configuration and dimensions and will increase the spectrum of the diseases that could be managed by this surgical approach.

In some patients it will be possible to introduce 2 cannulas in the stomach allowing bimanual percutaneous handling of gastric lesions under endoscopic control which can revolutionize intragastric surgery.

The cannula can also be designed to allow another scope to go inside the stomach and the procedure could be shared by two endoscopists (one oral endoscopist and the other transabdominal endoscopist ). One will be the main endoscopist and the other will be an assistant endoscopist creating a new way of endoscopic practice very similar to the situation in laparoscopic surgery.

The list of the diseases that could benefit from this technique includes superficial gastric tumors that could be managed by mucosal resection, fundal varices, pancreatic cysts, large mucosal and submucosal tumors, and certain cases of gastroduodenal bleeding.

In general the new approach will allow elaborate intragastric maneuvers to be done. These interventions could be done in endoscopy unit under sedation and local anesthesia without the need for OR setup or general anesthesia.

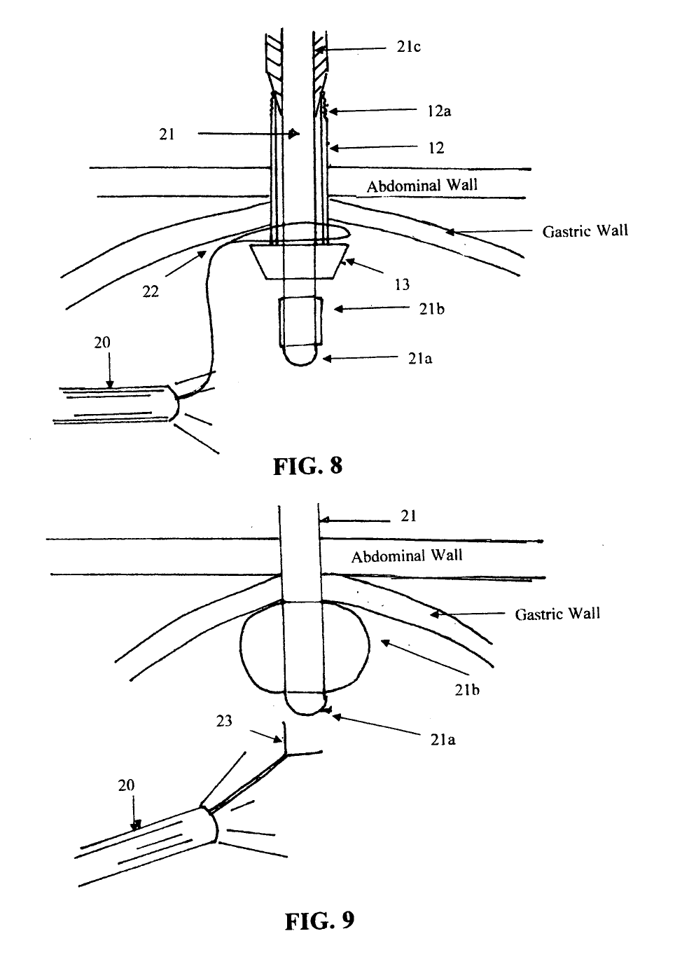

FIG. 8 is a sectional view in the extraction catheter used for removal of transgastric part of the cannula with solid-type internal gastric bolster according to one aspect of the present invention comprising essentially of a shaft (21) ending in a blunt distal end (21a) containing an inflatable balloon (21b), the diameter of this catheter will be suitable to that of the transgastric part of the and will end proximally in a wider segment (21c) to prevent gas leakage during extraction.

The extraction method of the transgastric part of the PEIGS cannula with a solid-type internal gastric bolster according to one embodiment of the present invention entails:
1-The external part of the PEIGS cannula as well as the external bolster and adhesive sheet are removed, while the gas leak controlled by assistant's finger.
2-A snare (22) is passed endoscopically to encircle the junction between the cylindrical part of the transgastric part of the PEIGS cannula (12) and the internal gastric bolster (13).
3-Then the extraction catheter is passed through the transgastric part while its balloon is deflated, the thicker proximal part (21c) will control gas leak at this time.
4-Then the transgastric part of the cannula is snared and removed through the mouth over the extraction catheter that is then inflated and pulled out to keep stomach retracted towards the abdominal wall.
5-Finally, with the endoscopist ready to deploy the endoclips (23) to the gastric defect, the balloon is deflated and the extraction catheter removed through the skin.

The use of this catheter during extraction will be more important in removing cannulas of larger diameter as the resultant defects will be larger while it may be possible to remove smaller cannulas without this catheter.

FIG. 9 is a sectional view in the extraction catheter with the balloon inflated and the cannula removed. This catheter is now used to maintain gentle traction on the stomach wall to seal the gastric defect to prevent escaping of the intragastric air and hence collapse of the stomach, that will make closure of the gastric defect difficult, and also prevent leakage of gastric contents and/or gastric wall bleeding. This will be essential during the time the endoscope is temporary removed together with the transgastric part of the cannula as it is clearly too large to pass through its biopsy channel, and during preparation for applying endoclips to the gastric defect.

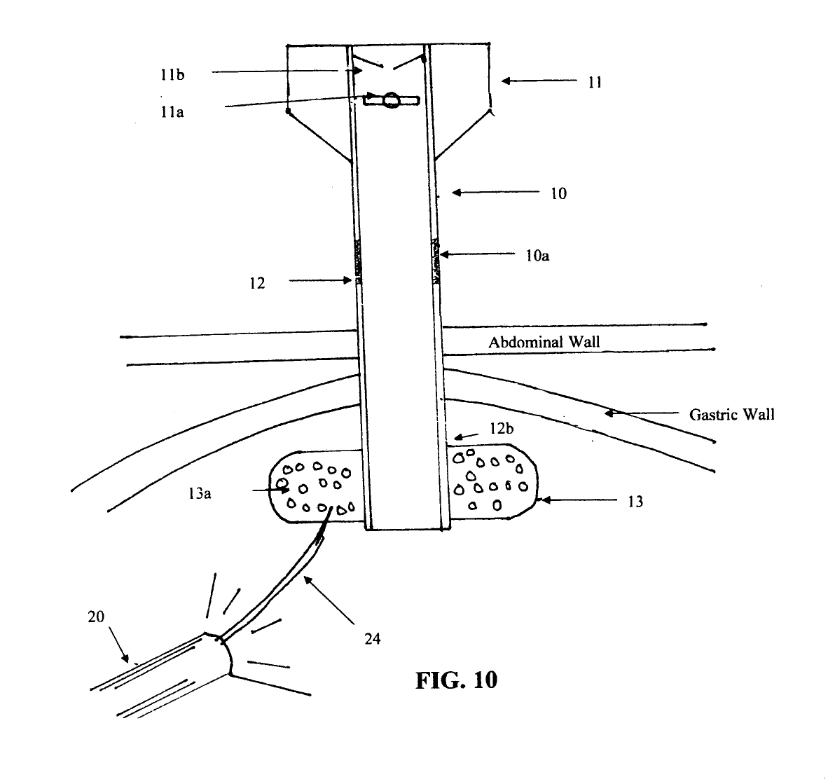

FIG. 10 shows the assembled cannula with a balloon-type internal gastric bolster in situ, with the endoscopist ready to rupture the balloon with an endoscopic needle (24). Here again the cannula should be kept in place to seal gastric hole till the endoscopist ready to apply endoclips, at this time the whole cannula will be removed through the skin.

Although the invention has been described in relation to its application in gastric surgery, it can also be used in similar way in treating colonic diseases affecting segments that could be accessed endoscopically and percuteneously.

Having thus described the invention, what we desire to claim and secure by Letters Patent is.

1- An instrument to allow endoscopic percutaneous access to the stomach cavity allowing elaborate surgical interventions comprising a two-part cannula, that apparatus is named percutaneous endoscopic intragastric surgery (PEIGS) cannula consisting of:
a-An external part consisting of a cylindrical segment ending in a proximal larger part containing a gas inlet to allow gastric insufflation and a system to control gas and at the same time, allow instruments to go in and out.
b-A transgastric part consisting of a cylindrical segment ending distally in an dilated internal gastric bolster.

2- The transgastric part of the said (PEIGS) cannula according to claim 1 further having a detachable introduction system comprising a tapering tube ending in a wire loop.

3- The instrument according to claim 1, further comprising an external bolster that together with the internal gastric bolster will control the axial movement of the assembled cannula.

4- The instrument according to claim 1, further comprising an extraction catheter consisting essentially of a shaft ending in a blunt end containing an inflatable balloon to be used during removal of cannulas with solid-type internal gastric bolster.

5- The cylindrical segment of external part of the apparatus according to claim 1, further ending distally in a segment adapted to unite in a secure air-tight fashion with the proximal end of the transgastric part of the cannula.

6- The external part of the said (PEIGS) cannula according to claim 1, will be made from plastic or plastic-like material or from metal.

7- The metal external part of the said (PEIGS) cannula according to claim 5, wherein it can be reusable

8- The cylindrical segments of the external and transgastric parts of the said (PEIGS) cannula according to claim 1, wherein the internal diameter ranges from 2-15 mm.

9- The cylindrical segment of the transgastric part of the said (PEIGS) cannula according to claim 1 wherein the length ranges from 5-8 cm.

10- The length of the cylindrical segment of the transgastric part of the said (PEIGS) cannula according to claim 8, wherein the length is more than 8 cm to fit obese patients.

11- The internal gastric bolster segment of the transgastric part of the said (PEIGS) cannula according to claim 1, further can be either solid-type made from soft deformable material or balloon-type filled with an inert fluid.

12- The cylindrical segment of transgastric part of the apparatus according to claim 1 further ending proximally in a segment adapted to unite in a secure way to the introduction system and also to unite in a secure air-tight fashion with the distal end of the external part of the cannula.

13- A method for introduction and removal of the said (PEIGS) cannula including endoscopically assisted introduction of the transgastric part by pull or push methods followed by fixation of the external part to assemble the complete cannula.

14- The method for introduction and removal of the said (PEIGS) cannula according to claim 13, further including removal of the transgastric part with solid-type internal gastric bolster at the end of the procedure using extraction catheter to prevent gas escape, gastric content leakage or gastric wall bleeding till endoscopist is ready to deploy endoclips to the gastric defect

15- The method for introduction and removal of the said (PEIGS) cannula according to claim 13, further including removal of the transgastric part with balloon-type internal gastric bolster by rupturing the balloon by endoscopic needle and removing the cannula though the skin

16- A method for using the said two-part (PEIGS) cannula to allow percutaneous access to the stomach using available laparoscopic instruments as well as specially designed instruments.

17- A method for using the said (PEIGS) cannula according to claim 16, wherein it is used to allow two endoscopes to pass to the stomach one transorally and the other through a (PEIGS) cannula allowing two endoscopists to share a procedure in a manner similar to laparoscopic surgery.

18- The method for using the said (PEIGS) cannula according to claim 16 to allow percutaneous needlescopic intragastric surgery by using 2-3 mm cannulas (PEIGS) cannulas.

19- The method for using the said (PEIGS) cannula according to claim 16, wherein two cannulas are introduced in the stomach to allow bimanual percutaneous intragastric surgery under endoscopic control.
